(19) United States
(12) Patent Application Publication
(10) Pub. No.: US 2026/0108273 A1
(43) Pub. Date: Apr. 23, 2026

(54) CARDIAC PACEMAKER ELECTRODE WIRE DELIVERY SHEATH

(71) Applicant: KOSSEL MEDTECH (SUZHOU) CO., LTD., Suzhou, Jiangsu (CN)

(72) Inventors: Qing HU, Suzhou, Jiangsu (CN); Xingzhen PAN, Suzhou, Jiangsu (CN)

(21) Appl. No.: 19/119,375

(22) PCT Filed: Mar. 3, 2024
(86) PCT No.: PCT/CN2024/079778

§ 371 Date: Apr. 8, 2025

(30) Foreign Application Priority Data
Mar. 3, 2023 (CN) 202310193884.6

(51) Int. Cl. A61B 17/34; A61B 17/00; A61B 17/32; A61B 17/3209; A61N 1/05

(52) U.S. Cl. CPC A61B 17/3468; A61B 17/00234; A61B 17/3209; A61N 1/0587; A61B 2017/00243; A61B 2017/00336; A61B 2017/00353; A61B 2017/00526; A61B 2017/00871; A61B 2017/320052

(57) ABSTRACT

Embodiments of the present specification provide a cardiac pacemaker electrode lead delivery sheath including a sheath tube including at least a first tube section provided at a distal end, the first tube section having a first curved section and a second curved section, the first curved section is provided at the distal end of the first tube section. Wherein the included angle between the first bending surface of the first curved section and the second bending surface of the second curved section is not less than 45°.

### CROSS REFERENCE

This application claims priority to Chinese Application with the Application No. 202310193884.6 filed on Mar. 3, 2023, the entire contents of which are incorporated herein by reference.

### TECHNICAL FIELD

The present specification relates to the technical field of medical device, and in particular to a cardiac pacemaker electrode lead delivery sheath.

### BACKGROUND

A cardiac pacemaker is an implanted electronic therapy device that delivers electrical pulses powered by a battery through a pulse generator. These impulses are conducted via electrode leads to stimulate the myocardial tissue contacted by the electrodes, thereby inducing cardiac excitation and contraction. This mechanism aims to address cardiac dysfunction resulting from certain arrhythmias. One of the most critical steps in cardiac pacemaker implantation is the implantation of cardiac pacemaker electrode leads.

In clinical procedures, the physiology of selective site pacing, such as interventricular septum, conduction bundle, and atrial septum pacing, has been demonstrated in various studies. However, since the current electrode lead delivery sheath can only achieve interventricular septum or conduction bundle pacing, it cannot fully achieve selective site pacing; or, problems such as being able to fully achieve selective site pacing but requiring the implantation of a special electrode lead rather than other conventional electrode leads.

Based on this, it is desirable to provide a cardiac pacemaker electrode lead delivery sheath to address electrode lead compatibility issues to fully achieve selective pacing.

### SUMMARY

One or more embodiments of the present specification provide a cardiac pacemaker electrode lead delivery sheath including a sheath tube including at least a first tube section provided at a distal end, the first tube section having a first curved section and a second curved section, the first curved section is provided at the distal end of the first tube section; wherein the included angle between the first bending surface of the first curved section and the second bending surface of the second curved section is not less than 45°.

In some embodiments, the first bending surface is perpendicular to the second bending surface.

In some embodiments, the hardness of the first curved section is less than the hardness of the second curved section.

In some embodiments, the second curved section has a curved radius ranging from 18 cm to 70 cm.

In some embodiments, the proximal end point, distal end point and maximum bending point of the second curved section are interconnected to form a first reference triangle; in the first reference triangle, the length of the first connecting line is less than the length of the second connecting line, and the value of the first included angle at the maximum bending point ranges from 80° to 120°; the first connecting line is a connecting line between the proximal end point and the maximum bending point, and the second connecting line is a connecting line between the distal end point and the maximum bending point.

In some embodiments, the proximal end point, distal end point and most distal bending point of the second curved section are interconnected to form a second reference triangle; in the second reference triangle, the length of the third connecting line is less than the length of the fourth connecting line, and the value of the second included angle at the most distal bending point ranges from 90° to 130°; the third connecting line is a connecting line between the proximal end point and the most distal bending point, and the fourth connecting line is a connecting line between the distal end point and the most distal bending point.

In some embodiments, the first tube section includes at least a braid, the braid is made from braided wires, a diameter of the braided wire of the second curved section ranges from 0.04 mm to 0.06 mm, and a braid density of the second curved section ranges from 50 to 70.

In some embodiments, the second curved section has a curved radius ranging from 10 cm to 15 cm.

In some embodiments, the proximal end point, distal end point and maximum bending point of the second curved section are interconnected to form a first reference triangle; in the first reference triangle, the length of the first connecting line is less than the length of the second connecting line, and the value of the first included angle at the maximum bending point ranges from 80° to 120°; the first connecting line is a connecting line between the proximal end point and the maximum bending point, and the second connecting line is a connecting line between the distal end point and the maximum bending point.

In some embodiments, the proximal end point, distal end point and most distal bending point of the second curved section are interconnected to form a second reference triangle; in the second reference triangle, the length of the third connecting line is less than the length of the fourth connecting line, and the value of the second included angle at the most distal bending point ranges from 90° to 130°; the third connecting line is a connecting line between the proximal end point and the most distal bending point, and the fourth connecting line is a connecting line between the distal end point and the most distal bending point.

In some embodiments, the first tube section includes at least a braid, the braid is made from braided wires, a diameter of the braided wire of the second curved section ranges from 0.05 mm to 0.07 mm, and a braid density of the second curved section ranges from 60 to 80.

In some embodiments, the second curved section adopts a double-wire braid.

In some embodiments, the first curved section has a curved radius ranging from 5 cm to 8 cm.

In some embodiments, the sheath tube further includes a second tube section and a third tube section, the second tube section being located in the middle of the sheath tube, the third tube section is located more proximally than the second tube section, the hardness of the second tube section is greater than the hardness of the first tube section and less than the hardness of the third tube section.

In some embodiments, both the second tube section and the third tube section include at least a braid, the braid is braided from a braided wire, and the braid density of the second tube section is less than the braid density of the third tube section.

In some embodiments, the delivery sheath further includes a sheath base, the sheath tube further includes a cut tube section provided at a proximal end and an implant tube section provided at a distal end of the cut tube section, the first tube section is located at a distal end of the implant tube section; the cut tube section includes a connecting region and a buffer region provided from proximal to distal along the axial direction of the sheath tube, the sheath tube is connected to the sheath base by the connecting region, the buffer region is configured to provide cutting resistance between the sheath tube and the implant tube section.

In some embodiments, the cutting resistance provided by the buffer region decreases from proximal to distal along the axial direction of the sheath tube.

In some embodiments, the buffer region includes a first raised structure provided on an inner wall of the buffer region.

In some embodiments, the thickness of the first raised structure decreases from proximal to distal along the axial direction of the sheath tube.

In some embodiments, the first raised structure includes a cutting guide slot having a width that decreases from proximal to distal along the axial direction of the sheath tube.

In some embodiments, the first raised structure includes a plurality of raised portions spaced along an axial direction of the sheath tube.

In some embodiments, the buffer region includes a second raised structure provided on an inner wall of the buffer region, the thickness of the second raised structure decreasing from proximal to distal along the axial direction of the sheath tube.

In some embodiments, the sheath base includes a side branch, a handle, and a sheath base body, the handle connected to the sheath base body, one end of the side branch communicating with the sheath base body through the handle, the other end of the side branch having a valve, the valve having at least one connector connected thereto.

In some embodiments, the sheath base body is provided with a channel along the axial direction of the sheath tube, one end of the side branch communicating with the channel, the channel configured to deliver a medical device; the sheath base body has a thin-walled structure along the axial direction of the sheath tube, and a sidewall of the sheath base body is provided with a clamping block.

In some embodiments, the delivery sheath further includes a sheath cap including a sheath cap body provided with a device channel in an axial direction of the sheath tube, a sidewall of the sheath cap body is provided with a clamping slot, the clamping slot is clamped with the clamping block of the sheath base body.

In some embodiments, the delivery sheath further includes a hemostasis valve provided within the channel of the sheath base body; the hemostasis valve is provided with a circular hole structure for passing the medical device and an elastic structure provided at a distal end of the circular hole structure and configured to open or close the circular hole structure.

In some embodiments, the material of the first curved section includes a developing material.

### BRIEF DESCRIPTION OF DRAWINGS

The present specification will further be described by way of exemplary embodiments, which will be described in detail with reference to the accompanying drawings. These embodiments are not limiting, and in these embodiments, like numbers refer to like structures, wherein:

FIG. 1 is a schematic view of the structure of a cardiac pacemaker electrode lead delivery sheath according to some embodiments of the present specification;

FIG. 2 is an exemplary schematic view of multiple stereo curved shapes of a first tube section according to some embodiments of the present specification;

FIG. 3 is an exemplary schematic view of the first reference triangle and the second reference triangle according to some embodiments of the present specification;

FIG. 4 is a schematic view of the structure of a sheath tube structure according to some embodiments of the present specification;

FIG. 5 is an exemplary schematic view of the braid according to some embodiments of the present specification;

FIG. 6 is an exemplary schematic view of the braid according to further embodiments of the present specification;

FIG. 7 is a schematic view of the structure of a connection between the sheath base and the sheath tube according to some embodiments of the present specification;

FIG. 8 is a schematic view of the structure of a connection between the sheath base and the sheath tube according to further embodiments of the present specification;

FIG. 9 is a schematic view of the structure of a sheath base according to some embodiments of the present specification;

FIG. 10 is a schematic view of the structure that a sheath base body connected to a handle according to some embodiments of the present specification;

FIG. 11 is a schematic view of the structure of a sheath cap according to some embodiments of the present specification;

FIG. 12 is a schematic view of the structure of a hemostasis valve according to some embodiments of the present specification;

FIG. 13 is a schematic view of the structure of opening an outlet of a hemostasis valve according to some embodiments of the present specification; and

FIG. 14 is a schematic view of the structure of closing an outlet of a hemostasis valve according to some embodiments of the present specification.

In the figures:
1, sheath tube, 11, first tube section, 111, first curved section, 112, second curved section, 113, braid, 114, outer layer, 115, inner layer, 12, second tube section, 13, third tube section, 14, cut tube section, 141, connecting region, 142, buffer region, 1421, first raised structure, 14211, cutting guide slot, 1422, second raised structure, 14221, first raised portion, 14222, second raised portion, 15, implant tube section;
2, sheath base, 21, side branch, 211, valve, 22, handle, 23, sheath base body, 231, channel, 232, thin-walled structure, 233, clamping block;
3, sheath cap, 31, device channel, 32, clamping slot;
4, hemostasis valve, 41, circular hole structure, 411, first circular hole, 412, second circular hole, 42, elastic structure, 43, reinforcing rib.

### DETAILED DESCRIPTION

In order that the manner in which the embodiments of the present specification are recited in detail, a brief description of the drawings that accompany the detailed description can be briefly described as follows. It is obvious that the figures in the following description are only examples or embodiments of the present specification, and that the present specification can be applied to other similar situations according to these figures without inventive effort for a person skilled in the art. Unless otherwise apparent from the context of the language, or stated otherwise, like reference numbers in the figures refer to like structures or operations.

It should be understood that a “system”, “device”, “unit”, and/or “module”, as used herein, is one method for distinguishing between different levels of different components, elements, components, portions, or assemblies. However, other words may be substituted by other expressions if they achieve the same purpose.

As used in the present specification and the appended claims, the terms “a”, “an”, “said” and/or “the” are not intended to be exhaustive or to limit the invention to the precise form disclosed, and may include plural references unless the context clearly dictates otherwise. In general, the terms “comprise” and “include” are intended to cover only those steps and elements that are explicitly recited, but that do not constitute an exclusive list, and that a method or apparatus may include other steps or elements.

Arrhythmia refers to the general term of irregular, fast, or slow heartbeat caused by an abnormal cardiac electrical conduction system. The treatment methods for arrhythmia include drug therapy and non-drug therapy. Drug therapy can only control heart rhythm to a certain extent. Pacemaker implantation is currently the main treatment for bradyarrhythmia. The most critical step in cardiac pacemaker implantation (hereinafter referred to as implantation) is the implantation of cardiac pacemaker electrode lead.

In clinical surgery, the implantation of a cardiac pacemaker electrode lead shall be guided and supported by an auxiliary sheath tube, and the specific operation steps are as follows: firstly, the sheath is inserted into the cardiac cavity, and then the electrode lead is introduced into the cardiac cavity along the sheath tube. After the fixation of the electrode is completed, it is necessary to withdraw the sheath tube from the body, but since other test equipment is provided at the other end connected with the electrode, it will interfere with the withdrawal of the sheath tube, so it is necessary to cut the sheath in an axial direction to complete the whole withdrawal of sheath tube. During cutting, the operator (e.g. doctor) needs to precisely control the force, which puts higher demands on the operator and may cause unnecessary injury to the patient due to the difficulty in controlling the force.

In addition, the physiology of selective site pacing, such as interventricular septum, conduction bundle, and atrial septum pacing, has been demonstrated in various studies. However, the current electrode lead delivery sheaths only achieve interventricular septum or conduction bundle pacing and cannot fully achieve selective site pacing; or, problems such as being able to fully achieve selective site pacing but requiring the implantation of a special electrode lead rather than other conventional electrode leads.

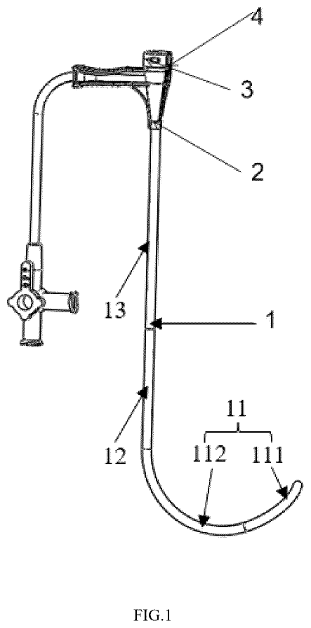

FIG. 1 is a schematic view of the structure of a cardiac pacemaker electrode lead delivery sheath according to some embodiments of the present specification. As shown in FIG. 1, some embodiments of the present specification provide a cardiac pacemaker electrode lead delivery sheath (hereinafter referred to as a delivery sheath), including a sheath tube 1 including at least a first tube section 11 provided at a distal end, the first tube section 11 is provided with a first curved section 111 and a second curved section 112, the first curved section 111 being located at the distal end of the first tube section 11. Wherein the included angle between the first bending surface of the first curved section 111 and the second bending surface of the second curved section is not less than 45°.

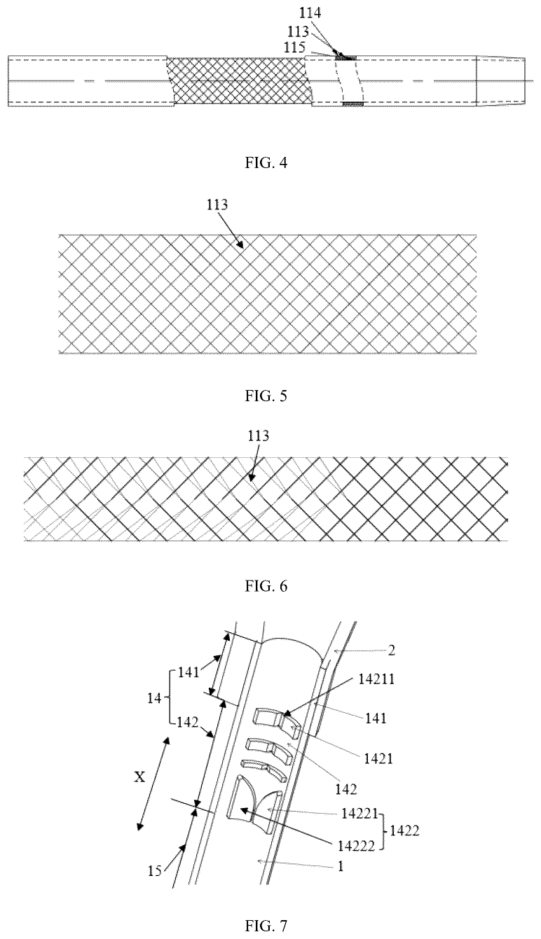

Sheath tube 1 refers to the tubular structure used to deliver the electrode lead. In some embodiments, the sheath tube 1 may be a one-piece structure or a split structure. In some embodiments, the sheath tube 1 may be formed by a rheological compounding process. In some embodiments, the sheath tube 1 may be divided into multiple tube sections, such as 3 sections, 5 sections, 10 sections, etc. depending on the circumstances. For example, the sheath tube 1 may include a first tube section 11, a second tube section 12, and a third tube section 13 as described hereinafter. In some embodiments, all or part of the tube section of the sheath tube 1 includes at least a braid. For further details of the braid, reference is made to FIG. 4 to FIG. 6 and the associated description thereof.

The first tube section 11 refers to the sheath tube section at the distal end of the sheath tube 1. The first curved section 111 is a sheath tube section at the distal end of the first tube section 11 and the second curved section 112 is a sheath tube section at the proximal end of the first tube section 11. The proximal end refers to the end close to the operator; the distal end refers to the end far from the operator. In some embodiments, the first curved section 111 and the second curved section 112 may be connected to each other (as shown in FIG. 1) or may be spaced apart (e.g. other curved sections may be provided between the first curved section 111 and the second curved section 112).

In some embodiments, both the first curved section 111 and the second curved section 112 may be designed to be curved (i.e. the first tube section 11 is curved) to match the structural shape of the first tube section 11 to different chamber structures of the heart to enable better selective site pacing. In some embodiments, the different curved sections may be of different materials and the different curved sections may have different hardnesses. Therefore, the first curved section 111 and the second curved section 112 can be divided by the difference in hardness or material to determine the positions of both ends thereof, i.e. the respective starting end position and ending end position.

In some embodiments, the material of the first curved section 111 includes at least a developing material. The developing material means a material having a developing effect or a developing function. Exemplary developing materials include but are not limited to, bismuth subcarbonate (Bi2O2CO3), barium sulfate (BaSO4), tungsten (W), and the like.

It will be appreciated that, since the first curved section 111 is located at the distal end of the first tube section 11, i.e. the distal end (i.e. the tip) of the sheath tube 1, when the material of the first curved section 111 is filled or added with a developing material, the tip of the sheath tube 1 can be made to have a developing property, so as to track the position of the tip of the sheath and achieve precise positioning of the tip of the sheath, thereby ensuring a precise operation during the operation.

The first bending surface is a plane in which a triangle is formed by interconnecting any point (e.g. a proximal end point, a distal end point) on both ends of the first curved section 111 and the maximum bending point or the most distal bending point. For example, the first bending surface may be a horizontal surface. The second bending surface is a plane in which a triangle is formed by interconnecting any point (e.g. a proximal end point, a distal end point) on both ends of the second curved section 112 and the maximum bending point or the most distal bending point. For example, the second bending surface may be a vertical surface. For further details of the proximal end point, the distal end point, the maximum bending point, and the most distal bending point, reference is made to FIG. 3 and associated description thereof.

In some embodiments, the first bending surface of the first curved section 111 has an included angle with the second bending surface of the second curved section 112. That is to say, the first curved section 111 and the second curved section 112 are in different planes, and the first curved section 111 and the second curved section 112 are curved in different planes, namely, the first tube section 11 is in a stereo curved shape.

In some embodiments, the included angle between the first bending surface and the second bending surface is not less than 45°. In some embodiments, the included angle between the first bending surface and the second bending surface may be between 45° and 54°. In some embodiments, the included angle between the first bending surface and the second bending surface may be between 55° and 64°. In some embodiments, the included angle between the first bending surface and the second bending surface may be between 65° and 74°. In some embodiments, the first bending surface is perpendicular to the second bending surface, i.e. the included angle between the first bending surface and the second bending surface is 90°.

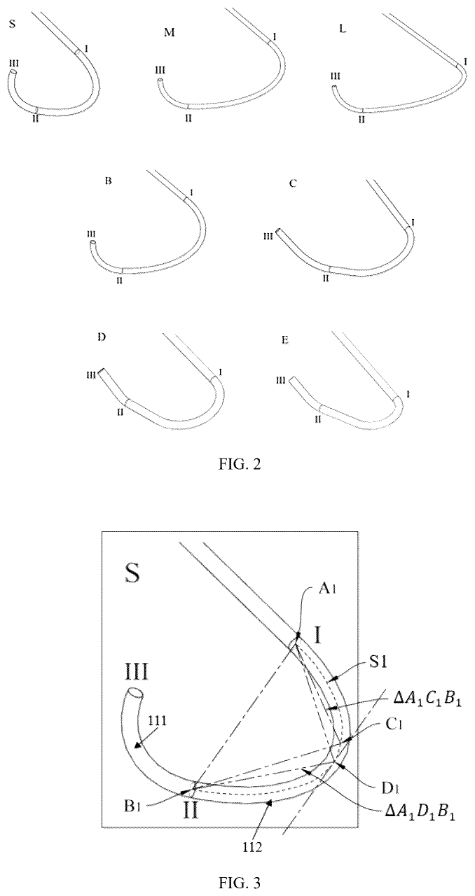

For more information about the sheath tube, please refer to the description in other parts of the present specification (such as the relevant description in FIG. 2 to FIG. 8).

In some embodiments of the present specification, selective site pacing is achieved by designing the distal end of the sheath tube to be in stereo curved shape so that it can better match different chamber configurations of the heart and establish access for the delivery electrode lead so that the tip of the electrode lead can reach the targeted pacing site.

The different curved sections of the first tube section 11 have different hardnesses. In some embodiments, the second curved section 112 is made of a material having a higher hardness because the second curved section 112 needs to be pre-formed into a pre-set curved shape and maintain the shape; while the first curved section 111 is located at the distal end (i.e. top end) of the sheath tube 1, it mainly plays a guiding role during the implantation operation, not only needs to adapt to the shape change of blood vessel, but also needs to avoid damaging the blood vessel wall, so that the first curved section 112 is made of a material with lower hardness. That is, the hardness of the first curved section 111 is less than the hardness of the second curved section 112.

In some embodiments, in order to better meet the deformation requirements of the first curved section 111 to ensure its functionality and safety, the hardness range of the first curved section 111 may be defined. In some embodiments, the hardness of the first curved section 111 may range from 30D to 35D. In some embodiments, the hardness of the first curved section 111 may range from 25D to 30D. In some embodiments, the hardness of the first curved section 111 may range from 35D to 40D. By limiting the hardness of the first curved section 111 within a suitable range, the first curved section 111 can be adapted to various shape changes of a blood vessel, not only can better guide a medical device such as an electrode lead but also can effectively avoid damaging the vessel wall.

In some embodiments, a range of hardness of the second curved section 112 may be defined in order to better maintain the preset curved shape of the second curved section 112. In some embodiments, the hardness of the second curved section 112 may range from 40D to 45D. In some embodiments, the hardness of the second curved section 112 may range from 35D to 40D. In some embodiments, the hardness of the second curved section 112 may range from 45D to 50D. By limiting the hardness of the second curved section 112 to an appropriate range, the second curved section 112 can better maintain the preset curved shape and provide sufficient support force for supporting the atrium or ventricle to establish access for medical devices such as electrode leads.

In some embodiments, a range of ratios of the hardness of the first curved section 111 to the hardness of the second curved section 112 may be defined in order to simultaneously meet the requirements of implant guidance and maintain the preset curved shape. In some embodiments, the ratio of the hardness of the first curved section 111 to the hardness of the second curved section 112 may range from 0.67 to 0.86. In some embodiments, the ratio of the hardness of the first curved section 111 to the hardness of the second curved section 112 may range from 0.70 to 0.80. In some embodiments, the ratio of the hardness of the first curved section 111 to the hardness of the second curved section 112 may range from 0.75 to 0.85. In some embodiments, the ratio of the hardness of the first curved section 111 to the hardness of the second curved section 112 may range from 0.65 to 0.80. In some embodiments, the ratio of the hardness of the first curved section 111 to the hardness of the second curved section 112 may range from 0.70 to 0.90. It will be appreciated that by defining a range of ratios of the hardness of the first curved section 111 to the hardness of the second curved section 112 such that the hardness requirements of both the hardness of the first curved section 111 and the hardness of the second curved section 112 are met, it is not only possible to ensure that the first curved section 111 does not damage the vessel wall when guided, but it is also advantageous to maintain the predetermined curved shape of the second curved section 112 to provide sufficient support.

In some embodiments, as shown in FIG. 1, the sheath tube 1 may also include a second tube section 12 located in the middle of the sheath tube 1 and a third tube section 13 located more proximally than the second tube section 12. That is, the second tube section 12 is located between the first tube section 11 and the third tube section 13.

The second tube section 12 refers to a sheath tube section located in the middle of the sheath tube 1, and the third tube section 13 refers to a sheath tube section located at the proximal end of the sheath tube 1. In some embodiments, the second tube section 12 may directly connect the first tube section 11 with the third tube section 13. In some embodiments, the second tube section 12 may also indirectly connect the first tube section 11 with the third tube section 13 through other tube sections.

In some embodiments, since the third tube section 13 is located more proximally than the second tube section 12, the operator needs to advance the sheath tube 1 along the third tube section 13 into the vessel during the implantation procedure, so the third tube section 13 is made of a material with a higher hardness; while the second tube section 12 is located between the first tube section 11 and the third tube section 13, in order to prevent the sheath tube 1 from bending due to abrupt changes in hardness, the second tube section 12 may be made of a material with medium hardness.

In some embodiments, the hardness of the second tube section 12 is greater than the hardness of the first tube section 11 and less than the hardness of the third tube section 13. That is, the hardness of the sheath tube 1 decreases from the proximal end to the distal end in the axial direction of the sheath tube 1. The axial direction of the sheath tube 1 refers to the extending direction of the centerline of the sheath tube 1.

In some embodiments, the hardness of the second tube section 12 ranges from 50D to 65D, and the hardness of the third tube section 13 ranges from 67D to 72D. In some embodiments, the hardness of the second tube section 12 ranges from 50D to 55D, and the hardness of the third tube section 13 ranges from 67D to 68D. In some embodiments, the hardness of the second tube section 12 ranges from 55D to 60D, and the hardness of the third tube section 13 ranges from 69D to 70D. In some embodiments, the hardness of the second tube section 12 ranges from 60D to 65D, and the hardness of the third tube section 13 ranges from 71D to 72D.

It will be appreciated that by defining or selecting the hardness range of the second tube section 12 and the third tube section 13, the hardness of the second tube section 12 and the third tube section 13 can be within an appropriate range, and at the same time ensuring the pushing performance of the sheath tube 1, bending of the sheath tube 1 can be effectively avoided, thereby ensuring the practicality and safety of the delivery sheath during the implantation operation.

In some embodiments, the hardness of each section of the sheath tube 1 (e.g. first tube section 11, second tube section 12, and third tube section 13) is related not only to the material used for each section but also to its structure. Therefore, it is feasible to select or design the materials or structures (such as the braiding mode of the braid) used for each tube section of sheath tube 1, so as to ensure that the hardness of each tube section of sheath tube 1 is within the appropriate range. For further details of the braid, reference is made to FIG. 4 to FIG. 6 and the associated description thereof.

In some embodiments, the sheath tube 1 may further include a cut tube section provided at the proximal end and an implant tube section provided at the distal end of the cut tube section, and the first tube section 11, the second tube section 12 and the third tube section 13 may be part of the implant tube section. For further details of the cut tube section and implant tube section, reference is made to FIG. 7 and FIG. 8 and the associated description thereof.

In some embodiments, the delivery sheath may also include a sheath base within which the proximal end of the sheath tube 1 is at least partially provided. The sheath base refers to the structure used to manipulate electrode lead delivery. In some embodiments, the sheath tube 1 may be attached to the sheath base in a variety of ways. Exemplary attachment means may include clamping, bonding, etc. In some embodiments, the medical device may be introduced into the sheath tube 1 via a sheath base (e.g. a channel of a sheath base body) and delivered via the sheath tube 1 to a target chamber (e.g. the right atrium of the heart). Medical device refers to one or more devices required for implantation surgery. For example, the medical device may include a guidewire, a dilator, an electrode lead, etc.

In some embodiments, the sheath base may include a side branch, a handle, and a sheath base body, wherein the handle is connected to the sheath base body, one end of the side branch passes through the handle and communicates with the sheath base body, and the other end of the side branch is provided with a valve, and at least one connector is connected to the valve.

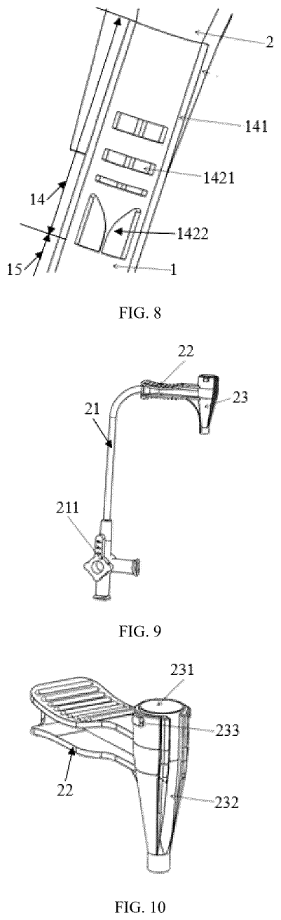

In some embodiments, the sheath base body is provided with a channel along an axial direction of the sheath base, one end of the side branch communicating with the channel, the channel configured to deliver a medical device. In some embodiments, the sheath base body has a thin-walled structure in the axial direction of the sheath tube, and the sidewall of the sheath base body is provided with a clamping block. For further details of the sheath base, reference is made to FIG. 9 and FIG. 10 and the associated description thereof.

In some embodiments, the delivery sheath may also include a sheath cap. The sheath cap is a structure that covers the proximal end of the sheath base body. In some embodiments, the sheath cap is provided with a device channel along the axial direction of the sheath tube and the sidewall of the sheath cap is provided with a clamping slot that is clamped with a clamping block of the sheath base body. In some embodiments, a sheath cap may be used to secure the hemostasis valve. For further details of the sheath cap, reference is made to FIG. 11 and the associated description thereof.

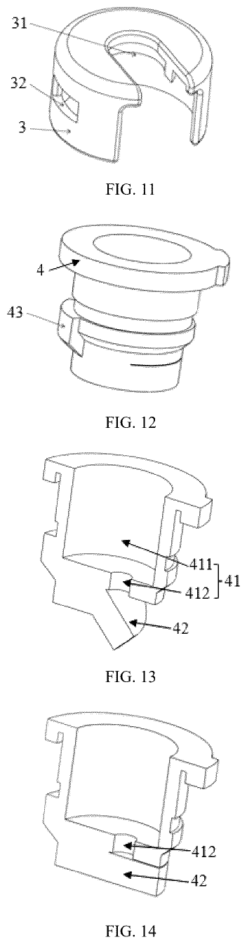

In some embodiments, the delivery sheath may also include a hemostasis valve. The hemostasis valve refers to a structure for preventing blood in a blood vessel from flowing out. In some embodiments, a hemostasis valve is provided within the channel of the sheath base body. In some embodiments, the hemostasis valve is provided with a circular hole structure for the passage of a medical device and an elastic structure provided at a distal end of the circular hole structure configured to open or close the circular hole structure. For further details of the hemostasis valve, reference is made to FIG. 12 to FIG. 14 and the associated description thereof.

As described above, the first tube section 11 may be designed to be in stereo curved shape in order to match different chamber configurations of the heart for better selective site pacing. It will be appreciated that the chamber structure of the heart is different from the pacing site, and the stereo curved shape of the matched first tube section 11 is also different. In some embodiments, the chamber structure of the heart may include a chamber shape of the heart and a chamber size of the heart.

FIG. 2 is an exemplary schematic view of multiple stereo curved shapes of a first tube section according to some embodiments of the present specification. By way of example only, as shown in FIG. 2, the stereo curved shapes of the first tube section 11 may include seven types, S, M, L, B, C, D, E, labeled S-type, M-type, L-type, B-type and C-type, D-type, and E-type, respectively. In the figure, the tube section between points II and III is a first curved section 111, and the tube section between points I and II is a second curved section 112. As can be seen from FIG. 2, the stereo curved shape of the first tube section 11 depends on the curving degree of the first 111 and second 112 curved sections, in particular the curving degree of the second curved section 112. In some embodiments, the curving degree may be characterized by a curved radius, the smaller the curved radius, the greater the curving degree.

In some embodiments, the curved radius of the second curved section 112 is relatively large because the second curved section 112 is primarily designed to support the atrium or ventricle and provide sufficient support to access the electrode lead; while the first curved section 111 is mainly used to guide the tip of the electrode lead to the target pacing site, the curved radius of the first curved section 111 is small.

In some embodiments, in order to enable the first tube section 11 to smoothly enter the target cavity while guiding the tip of the electrode lead to the target pacing site so as to realize selective site pacing, the stereo curved shape of the first tube section 11 used is different according to the cavity structure where different pacing sites are located. In some embodiments, since the size of the atrium is smaller than the size of the ventricle when the delivery sheath is used for interventricular septum, his bundle, and left bundle branch pacing, the tip of the sheath tube 1 (or the distal end of the first curved section 111) needs to reach the right ventricle, at this time, the curved radius of the second curved section 112 should be larger, and the stereo curved shape of the first tube section 11 may be any one of the S-type, M-type, L-type, B-type and C-type as previously described. When the delivery sheath is used for atrial septum pacing, the tip of the sheath tube 1 (or the distal end of the first curved section 111) needs to reach the right atrium, in this case, the curved radius of the second curved section 112 should be small, and the stereo curved shape of the first tube section 11 may be any one of D-type and E-type as previously described. It should be noted that for a delivery sheath for the same chamber shape (e.g. right atrium), the stereo curved shape of the first tube section 11 depends on the size of the chamber. For example, for a delivery sheath for use in the right atrium of a child, the stereo curved shape of the first tube section 11 may be S-shaped or the like.

In some embodiments, when the delivery sheath is used for interventricular septum, his bundle and left bundle branch pacing, for example, the stereo curved shape of the first tube section 11 is S-type, M-type, L-type, B-type and C-type, the curved radius of the second curved section 112 may range from 18 cm to 70 cm. In some embodiments, when the delivery sheath is used for interventricular septum, his bundle and left bundle branch pacing, for example, the stereo curved shape of the first tube section 11 is S-type, M-type, L-type, B-type, and C-type, the curved radius of the second curved section 112 ranges from 15 cm to 55 cm. In some embodiments, when the delivery sheath is used for interventricular septum, his bundle and left bundle branch pacing, for example, the stereo curved shape of the first tube section 11 is S-type, M-type, L-type, B-type and C-type, the curved radius of the second curved section 112 may range from 25 cm to 80 cm.

In some embodiments, the structural shape of the second curved section 112 may be characterized by constructing a triangle on the second curved section 112 and based on relevant data for the triangle. FIG. 3 is an exemplary schematic view of the first reference triangle and the second reference triangle according to some embodiments of the present specification. As shown in FIG. 3, when the delivery sheath is used for interventricular septum, his bundle, and left bundle branch pacing, for example, the stereo curved shape of the first tube section 11 is S-type, M-type, L-type, B-type, and C-type, the proximal end point A₁, the distal end point B₁ and the maximum bending point C₁ of the second curved section 112 are interconnected to form a first reference triangle ΔA₁C₁B₁. In the first reference triangle ΔA₁C₁B₁, the length of the first connecting line is less than the length of the second connecting line, and the value range of the first included angle ∠A₁C₁B₁ at the maximum bending point Ciranges from 80° to 120°. Wherein the first connecting line is a connecting line A₁C₁ between the proximal end point A₁ and the maximum bending point C₁, and the second connecting line is a connecting line B₁C₁ between the distal end point Bi and the maximum bending point C₁.

The center line of the second curved section 112 forms a first curve S1, and the proximal end point A₁ refers to a proximal end point of the first curve S1; the distal end point B₁ refers to the distal end point of the first curve S1; the maximum bending point C₁ refers to a point on the first curve S1 at which the curvature is maximum, or a point on the first curve S1 at which the curved radius is minimum.

The first included angle ∠A₁C₁B₁, which is the included angle at the maximum bending point C₁ in the first reference triangle ΔA₁C₁B₁, may reflect the curving degree or curved radius of the second curved section 112. For example, the smaller the angle of the first included angle ∠A₁C₁B₁ (such as an acute angle), the higher the curving degree of bending of the second curved section 112, the smaller the curved radius; the greater the angle of the first included angle ∠A₁C₁B₁ (e.g. obtuse angle), the lower the curving degree of the second curved section 112, the greater the curved radius.

In some embodiments, when the delivery sheath is used for interventricular septum, his bundle and left bundle branch pacing, for example, the stereo curved shape of the first tube section 11 is S-type, M-type, L-type, B-type, and C-type, the value of the first included angle ∠A₁C₁B₁ may also range from 80° to 90°. In some embodiments, when the delivery sheath is used for interventricular septum, his bundle and left bundle branch pacing, for example, the stereo curved shape of the first tube section 11 is S-type, M-type, L-type, B-type, and C-type, the value of the first included angle ∠A₁C₁B₁ may also range from 110° to 120°.

In some embodiments, since the area of the first reference triangle ΔA₁C₁B₁ can not only reflect the curving degree and the extension dimension of the second curved section 112 but also directly reflect the size of the two-dimensional space occupied by the first reference triangle ΔA₁C₁B₁. Therefore, the structural shape of the second curved section 112 can be adapted to different chamber structures of the heart by limiting the area of the first reference triangle ΔA₁C₁B₁.

In some embodiments, when the delivery sheath is used for interventricular septum, his bundle and left bundle branch pacing, for example, the stereo curved shape of the first tube section 11 is S-type, M-type, L-type, B-type, and C-type, the area of the first reference triangle ΔA₁C₁B₁ may range from 20 cm² to 35 cm². In some embodiments, when the delivery sheath is used for interventricular septum, his bundle and left bundle branch pacing, for example, the stereo curved shape of the first tube section 11 is S-type, M-type, L-type, B-type, and C-type, the area of the first reference triangle ΔA₁C₁B₁ may range from 17 cm² to 20 cm². In some embodiments, when the delivery sheath is used for interventricular septum, his bundle and left bundle branch pacing, for example, the stereo curved shape of the first tube section 11 is S-type, M-type, L-type, B-type, and C-type, the area of the first reference triangle ΔA₁C₁B₁ may range from 35 cm² to 40 cm².

It will be appreciated that when the delivery sheath is used for interventricular septum, his bundle and left bundle branch pacing, for example, the stereo curved shape of the first tube section 11 is S-type, M-type, L-type, B-type and C-type, the structural shape of the second curved section 112 can be adapted to different chamber structures of the heart by defining the area of the first reference triangle ΔA₁C₁B₁ within a suitable range, so as to lay the foundation for achieving selective site pacing.

In some embodiments, in addition to directly defining the area of the first reference triangle ΔA₁C₁B₁, the second curved section 112 structural shape ΔA₁C₁B₁ may be adapted to different chamber structures of the heart by defining the side lengths of the first reference triangle.

In some embodiments, when the delivery sheath is used for interventricular septum, his bundle and left bundle branch pacing, for example, the stereo curved shape of the first tube section 11 is S-type, M-type, L-type, B-type, and C-type, the length of the connecting line A₁B₁ between the proximal end point A₁ and the distal end point B₁ may range from 6 cm to 8 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the connecting line A₁B₁ between the proximal end point A₁ and the distal end point B₁ may range from 5 cm to 6 cm. In some embodiments, when the delivery sheath is used for interventricular septum, his bundle, and left bundle branch pacing, for example, the stereo curved shape of the first tube section 11 is S-type, M-type, L-type, B-type, and C-type, the length of the connecting line A₁B₁ between the proximal end point A₁ and the distal end point B₁ ranges from 8 cm to 10 cm.

In some embodiments, when the delivery sheath is used for interventricular septum, his bundle, and left bundle branch pacing, for example, the stereo curved shape of the first tube section 11 is S-type, M-type, L-type, B-type, and C-type, the length of the first connecting line A₁C₁ may range from 3 cm to 5 cm. In some embodiments, when the delivery sheath is used for interventricular septum, his bundle, and left bundle branch pacing, for example, the stereo curved shape of the first tube section 11 is S-type, M-type, L-type, B-type, and C-type, the length of the first connecting line A₁C₁ may range from 2 cm to 3 cm. In some embodiments, when the delivery sheath is used for interventricular septum, his bundle, and left bundle branch pacing, for example, the stereo curved shape of the first tube section 11 is S-type, M-type, L-type, B-type, and C-type, the length of the first connecting line A₁C₁ may range from 5 cm to 6 cm.

In some embodiments, when the delivery sheath is used for interventricular septum, his bundle and left bundle branch pacing, for example, the stereo curved shape of the first tube section 11 is S-type, M-type, L-type, B-type, and C-type, the length of the second connecting line B₁C₁ may range from 7 cm to 9 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the second connecting line B₁C₁ may range from 6 cm to 7 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the second connecting line B₁C₁ may range from 9 cm to 10 cm.

It will be appreciated that when the delivery sheath is used for interventricular septum, his bundle, and left bundle branch pacing, for example, when the stereo curved shape of the first tube section 11 is S-type, M-type, L-type, B-type, and C-type, the structural shape of the second curved section 112 can also be defined by defining the side length of the first reference triangle ΔA₁C₁B₁ within a suitable range, so that it can be adapted to different chamber structures of the heart, thereby laying the foundation for achieving selective site pacing.

In some embodiments of the present specification, when the delivery sheath is used for interventricular septum, his bundle and left bundle branch pacing, for example, when the stereo curved shape of the first tube section 11 is S-type, M-type, L-type, B-type and C-type, the curving degree of the second curved section 112 can be clearly characterized by constructing a first reference triangle ΔA₁C₁B₁ on the second curved section 112, wherein the first included angle ∠A₁C₁B₁ at the maximum bending point C₁; further combining the area and side length of the first reference triangle ΔA₁C₁B₁, the structural shape of the second curved section 112 and the matching degree of the different chamber structures of the heart can be more intuitively embodied. On this basis, by defining the first included angle ∠A₁C₁B₁, the area or the side length of the first reference triangle ΔA₁C₁B₁, it is possible to effectively ensure the matching degree of the first tube section 11 with the different chamber structures of the heart, thereby ensuring the functionality of the delivery sheath during the implantation procedure.

As shown in FIG. 3, the proximal end point A₁, the distal end point B₁, and the most distal bending point D₁ of the second curved section 112 are interconnected to form a second reference triangle ΔA₁D₁B₁. In the second reference triangle ΔA₁D₁B₁, the length of the third connecting line is less than the length of the fourth connecting line, and the value of the second included angle ∠A₁D₁B₁ at the most distal bending point D₁ ranges from 90° to 130°. The third connecting line is a connecting line A₁D₁ between the proximal end point A₁ and the most distal bending point D₁, and the fourth connecting line is a connecting line B₁D₁ between the distal end point B₁ and the most distal bending point D₁.

The most distal bending point D₁ refers to a point on the first curve most distal from a connecting line A₁B₁ connecting the proximal end point A₁ and the distal end point B₁. In some embodiments, the most distal bending point D₁ may be obtained by drawing a parallel line connecting line A₁B₁ and translating the parallel line to the right until tangent to the first curve, i.e. the tangent point of the parallel line to the first curve is the most distal bending point D₁. In some embodiments, the most distal bending point D₁ may or may not coincide with the maximum bending point C₁.

The second included angle ∠A₁D₁B₁ is the included angle at the most distal bending point D₁ in the second reference triangle ΔA₁B₁D₁, and can reflect the raised degree of the second curved section 112. For example, a smaller angle of the second included angle ∠A1D1B1 (e.g. an acute angle) indicates a higher degree of protrusion of the second curved section 112; the greater the angle of the second included angle ∠A₁D₁B₁ (e.g. obtuse angle), the less convex the second curved section 112.

In some embodiments, when the delivery sheath is used for interventricular septum, his bundle and left bundle branch pacing, for example, the stereo curved shape of the first tube section 11 is S-type, M-type, L-type, B-type, and C-type, the value of the second included angle ∠A₁D₁B₁ may range from 50° to 60°. In some embodiments, when the delivery sheath is used for interventricular septum, his bundle and left bundle branch pacing, for example, the stereo curved shape of the first tube section 11 is S-type, M-type, L-type, B-type, and C-type, the value of the second included angle ∠A₁D₁B₁ may range from 60° to 70°.

In some embodiments, the area of the second reference triangle ΔA₁D₁B₁ may be defined to adapt the structural shape of the second curved section 112 to different chamber configurations of the heart, since the area of the second reference triangle ΔA₁D₁B₁ may reflect not only the degree of protrusion of the second curved section 112, but also the size of the two-dimensional space occupied by the second reference triangle ΔA₁D₁B₁.

In some embodiments, when the delivery sheath is used for interventricular septum, his bundle and left bundle branch pacing, for example, the stereo curved shape of the first tube section 11 is S-type, M-type, L-type, B-type, and C-type, the area of the second reference triangle ΔA₁D₁B₁ may range from 25 cm² to 40 cm². In some embodiments, when the delivery sheath is used for interventricular septum, his bundle and left bundle branch pacing, for example, the stereo curved shape of the first tube section 11 is S-type, M-type, L-type, B-type, and C-type, the area of the second reference triangle ΔA₁D₁B₁ may range from 21 cm² to 25 cm². In some embodiments, when the delivery sheath is used for interventricular septum, his bundle and left bundle branch pacing, for example, the stereo curved shape of the first tube section 11 is S-type, M-type, L-type, B-type, and C-type, the area of the second reference triangle ΔA₁D₁B₁ may range from 40 cm² to 45 cm².

It will be appreciated that when the delivery sheath is used for interventricular septum, his bundle and left bundle branch pacing, for example, when the stereo curved shape of the first tube section 11 is S-type, M-type, L-type, B-type, and C-type, the structural shape of the second curved section 112 can also be defined by defining the area of the second reference triangle ΔA₁D₁B₁ within a suitable range, so that it can be adapted to different chamber structures of the heart, thereby laying the foundation for achieving selective site pacing.

In some embodiments, in addition to directly defining the area of the second reference triangle ΔA₁D₁B₁, the second curved section 112 structural shape may be adapted to different chamber structures of the heart by defining the side lengths of the second reference triangle ΔA₁D₁B₁.

In some embodiments, when the delivery sheath is used for interventricular septum, his bundle and left bundle branch pacing, for example, the stereo curved shape of the first tube section 11 is S-type, M-type, L-type, B-type, and C-type, the length of the third connecting line A₁D₁ may range from 7 cm to 9 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the third connecting line A₁D₁ may range from 6 cm to 7 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the third connecting line A₁D₁ may range from 9 cm to 10 cm.

In some embodiments, when the delivery sheath is used for interventricular septum, his bundle and left bundle branch pacing, for example, the stereo curved shape of the first tube section 11 is S-type, M-type, L-type, B-type, and C-type, the length of the fourth connecting line B₁D₁ may range from 3 cm to 5 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the fourth connecting line B₁D₁ may range from 2 cm to 3 cm. In some embodiments, when the delivery sheath is used for interventricular septum, his bundle and left bundle branch pacing, for example, the stereo curved shape of the first tube section 11 is S-type, M-type, L-type, B-type, and C-type, the length of the fourth connecting line B₁D₁ may range from 5 cm to 6 cm.

It will be appreciated that when the delivery sheath is used for interventricular septum, his bundle, and left bundle branch pacing, for example, when the stereo curved shape of the first tube section 11 is S-type, M-type, L-type, B-type, and C-type, the structural shape of the second curved section 112 can also be defined by defining the side length of the second reference triangle ΔA₁B₁D₁ within a suitable range, so that it can be adapted to different chamber structures of the heart, thereby laying the foundation for achieving selective site pacing.

In some embodiments of the present specification, when the delivery sheath is used for interventricular septum, his bundle and left bundle branch pacing, for example, when the stereo curved shape of the first tube section 11 is S-type, M-type, L-type, B-type and C-type, since the most distal bending point D₁ is farther from the connecting line A₁B₁ between the proximal end point Ai and the distal end point Bi than the maximum bending point C₁, the triangle formed by the most distal bending point Di and the proximal end point A₁ and the distal end point Bi can more accurately reflect the size of the space occupied by the second curved section 112. On this basis, by defining the second included angle ∠A₁D₁B₁, the area or the side length of the second reference triangle ΔA₁D₁B₁, it is possible to effectively ensure a degree of matching of the first tube section 11 with the different chamber structures of the heart, thereby further ensuring the functionality of the delivery sheath during the implantation procedure.

In some embodiments, when the delivery sheath is used for atrial septum pacing, e.g. the stereo curved shape of the first tube section 11 is D-type and E-type, the curved radius of the second curved section 112 may range from 10 cm to 15 cm. In some embodiments, when the delivery sheath is used for atrial septum pacing, e.g. the stereo curved shape of the first tube section 11 is D-type and E-type, the curved radius of the second curved section 112 may range from 8 cm to 10 cm. In some embodiments, when the delivery sheath is used for atrial septum pacing, e.g. the stereo curved shape of the first tube section 11 is D-type and E-type, the curved radius of the second curved section 112 may range from 15 cm to 20 cm.

As shown in FIG. 3, when the delivery sheath is used for atrial septum pacing, for example, when the stereo curved shape of the first tube section 11 is D-type and E-type, the proximal end point A₁, the distal end point B₁ and the maximum bending point C₁ of the second curved section 112 are interconnected to form a first reference triangle ΔA1C1B1. In the first reference triangle ΔA₁C₁B₁, the length of the first connecting line is less than the length of the second connecting line, and the value of the first included angle ∠A₁C₁B₁ at the maximum bending point C₁ ranges from 70° to 100°. Wherein the first connecting line is a connecting line A₁C₁ between the proximal end point A₁ and the maximum bending point C₁, and the second connecting line is a connecting line B₁C₁ between the distal end point B₁ and the maximum bending point C₁. Further details regarding the proximal end point, the distal end point, the maximum bending point, and the first included angle can be found in connection with the previous description of FIG. 3.

In some embodiments, when the delivery sheath is used for interventricular septum pacing, for example, the stereo curved shape of the first tube section 11 is D-type and E-type, the area of the first reference triangle ΔA₁C₁B₁ may range from 8 cm² to 20 cm². In some embodiments, when the delivery sheath is used for interventricular septum pacing, for example, the stereo curved shape of the first tube section 11 is D-type and E-type, the area of the first reference triangle ΔA₁C₁B₁ may range from 8 cm² to 12 cm². In some embodiments, when the delivery sheath is used for interventricular septum pacing, for example, the stereo curved shape of the first tube section 11 is D-type and E-type, the area of the first reference triangle ΔA₁C₁B₁ may range from 15 cm² to 20 cm².

It will be appreciated that when the delivery sheath is used for interatrial septum pacing, e.g. the stereo curved shape of the first tube section 11 is D-type and E-type, by defining the area of the first reference triangle ΔA₁C₁B₁ within a suitable range, i.e. the structural shape of the second curved section 112, so that it can be adapted to different chamber structures of the heart, thereby laying the foundation for achieving selective site pacing.

In some embodiments, when the delivery sheath is used for atrial septum pacing, e.g. the stereo curved shape of the first tube section 11 is D-type and E-type, the length of the connecting line A₁B₁ connecting the proximal end point A₁ and the distal end point B₁ may range from 3 cm to 5 cm. In some embodiments, when the delivery sheath is used for atrial septum pacing, e.g. the stereo curved shape of the first tube section 11 is D-type and E-type, the length of the connecting line A₁B₁ connecting the proximal end point A₁ and the distal end point B₁ may range from 2 cm to 3 cm. In some embodiments, when the delivery sheath is used for atrial septum pacing, e.g. the stereo curved shape of the first tube section 11 is D-type and E-type, the length of the connecting line A₁B₁ connecting the proximal end point A₁ and the distal end point B₁ may range from 5 cm to 6 cm.

In some embodiments, when the delivery sheath is used for atrial septum pacing, e.g. the stereo curved shape of the first tube section 11 is D-type and E-type, the length of the first connecting line A₁C₁ may range from 2 cm to 3 cm. In some embodiments, when the delivery sheath is used for atrial septum pacing, e.g. the stereo curved shape of the first tube section 11 is D-type and E-type, the length of the first connecting line A₁C₁ may range from 1 cm to 2 cm. In some embodiments, when the delivery sheath is used for atrial septum pacing, e.g. the stereo curved shape of the first tube section 11 is D-type and E-type, the length of the first connecting line A₁C₁ may range from 3 cm to 5 cm.

In some embodiments, when the delivery sheath is used for atrial septum pacing, e.g. the stereo curved shape of the first tube section 11 is D-type and E-type, the length of the second connecting line B₁C₁ may range from 3 cm to 4 cm. In some embodiments, when the delivery sheath is used for atrial septum pacing, e.g. the stereo curved shape of the first tube section 11 is D-type and E-type, the length of the second connecting line BICI may range from 2 cm to 3 cm. In some embodiments, when the delivery sheath is used for atrial septum pacing, e.g. the stereo curved shape of the first tube section 11 is D-type and E-type, the length of the second connecting line BICI may range from 4 cm to 6 cm.

It will be appreciated that when the delivery sheath is used for interatrial septum pacing, for example, when the stereo curved shape of the first tube section 11 is D-type and E-type, the structural shape of the second curved section 112 can also be defined by defining the side length of the first reference triangle ΔA₁C₁B₁ within a suitable range, so that it can be adapted to different chamber structures of the heart, thereby laying the foundation for achieving selective site pacing.

As shown in FIG. 3, when the delivery sheath is used for atrial septum pacing, for example, when the stereo curved shape of the first tube section 11 is D-type and E-type, the proximal end point A₁, the distal end point B₁ and the most distal bending point D₁ of the second curved section 112 are interconnected to form a second reference triangle ΔA₁D₁B₁. In the second reference triangle ΔA1D1B1, the length of the third connecting line is less than the length of the fourth connecting line, and the value of the second included angle ∠A₁D₁B₁ at the most distal bending point D₁ ranges from 80° to 110°. The third connecting line is a connecting line A₁D₁ between the proximal end point A₁ and the most distal bending point D₁, and the fourth connecting line is a connecting line B₁D₁ between the distal end point B₁ and the most distal bending point D₁. Further details regarding the second included angle of the most distal bending point can be found in connection with the previous description of FIG. 3.

In some embodiments, when the delivery sheath is used for interventricular septum pacing, for example, the stereo curved shape of the first tube section 11 is D-type and E-type, the area of the second reference triangle ΔA₁D₁B₁ may range from 10 cm² to 25 cm². In some embodiments, when the delivery sheath is used for interventricular septum pacing, for example, the stereo curved shape of the first tube section 11 is D-type and E-type, the area of the second reference triangle ΔA₁D₁B₁ may range from 10 cm² to 14 cm². In some embodiments, when the delivery sheath is used for interventricular septum pacing, for example, the stereo curved shape of the first tube section 11 is D-type and E-type, the area of the second reference triangle ΔA₁D₁B₁ may range from 20 cm² to 25 cm².

It will be appreciated that when the delivery sheath is used for interatrial septum pacing, e.g. the stereo curved shape of the first tube section 11 is D-type and E-type, the structural shape of the second curved section 112 can also be defined by defining the area of the second reference triangle ΔA₁D₁B₁ to be within a suitable range so that it can be adapted to different chamber structures of the heart, thereby laying the foundation for achieving selective site pacing.

In some embodiments, when the delivery sheath is used for atrial septum pacing, e.g. the stereo curved shape of the first tube section 11 is D-type and E-type, the length of the third connecting line A₁D₁ may range from 3 cm to 4 cm. In some embodiments, when the delivery sheath is used for atrial septum pacing, e.g. the stereo curved shape of the first tube section 11 is D-type and E-type, the length of the third connecting line A₁D₁ may range from 2 cm to 3 cm. In some embodiments, when the delivery sheath is used for atrial septum pacing, e.g. the stereo curved shape of the first tube section 11 is D-type and E-type, the length of the third connecting line A₁D₁ may range from 4 cm to 6 cm.

In some embodiments, when the delivery sheath is used for atrial septum pacing, e.g. the stereo curved shape of the first tube section 11 is D-type and E-type, the length of the fourth connecting line B₁D₁ may range from 2 cm to 3 cm. In some embodiments, when the delivery sheath is used for atrial septum pacing, e.g. the stereo curved shape of the first tube section 11 is D-type and E-type, the length of the fourth connecting line B₁D₁ may range from 1 cm to 2 cm. In some embodiments, when the delivery sheath is used for atrial septum pacing, e.g. the stereo curved shape of the first tube section 11 is D-type and E-type, the length of the fourth connecting line B₁D₁ may range from 3 cm to 5 cm.

It will be appreciated that when the delivery sheath is used for interatrial septum pacing, for example, when the stereo curved shape of the first tube section 11 is D-type and E-type, by defining the side length of the second reference triangle ΔA₁D₁B₁ within a suitable range, the structural shape of the second curved section 112 can also be defined so that it can be adapted to different chamber structures of the heart, thereby laying the foundation for achieving selective site pacing.

As described above, the stereo curved shape of the first tube section 11 mainly depends on the curving degree of the second curved section 112, whereby it can be considered that the curved radius of the first curved section 111 of the above-mentioned seven curve types is substantially the same. In addition, since the first curved section 111 is mainly used to guide the tip of the electrode lead to the target pacing site, the curved radius of the first curved section 111 is small.

In some embodiments, the curved radius of the first curved section 111 may range from 5 cm to 8 cm. In some embodiments, the curved radius of the first curved section 111 may range from 3 cm to 5 cm. In some embodiments, the curved radius of the first curved section 111 may range from 8 cm to 12 cm.

It will be appreciated that the curved radius of the first curved section 111 can be further defined based on defining the curved radius of the second curved section 112, to further ensure that the stereo curved shape of the first tube section 11 is adapted to different chamber configurations of the heart, thereby enabling the tip of the electrode lead to reach the targeted pacing site, thereby enabling selective site pacing.

FIG. 4 is a schematic view of the structure of a sheath tube structure according to some embodiments of the present specification; FIG. 5 is an exemplary schematic view of the braid according to some embodiments of the present specification; FIG. 6 is an exemplary schematic view of the braid according to further embodiments of the present specification. As shown in FIG. 4, all or part of the tube section of the sheath tube 1 includes a braid 113, an outer layer 114, and an inner layer 115. In some embodiments, outer layer 114, braid 113, and inner layer 115 may be formed into sheath tube 1 by a rheological compounding process.

In some embodiments, as shown in FIG. 4 to FIG. 6, the first tube section 11 includes at least a braid 113 that is braided from braided wire. The material of the braided wire may include a metal wire, such as a stainless steel wire.

In some embodiments, the first tube section 11 may also include an outer layer 114 and an inner layer 115, with the braid 113 located between the outer layer 114 and the inner layer 115. The outer layer 114 is located outside the braid 113 and is an outer wall layer of the sheath tube 1 (such as the first tube section 11); the inner layer 115 is located inside the braid 113 and is the inner wall layer of the sheath tube 1 (e.g. the first tube section 11). In some embodiments, the outer layer 114 and the inner layer 115 may be the same or different. For example, a flexible material such as Pebax may be used for the outer layer 114 to avoid damage to the patient's blood vessels during the implantation procedure; the inner layer 115 may be made of a lubricious material such as PTFE to reduce the resistance to the passage of a medical device such as an electrode lead so that the medical device can smoothly reach the target cavity.

In some embodiments, since the delivery sheath may come into contact with human tissues or organs such as blood vessels during the implantation operation, the first tube section 11 in a stereo curved shape, in particular the second curved section 112, can be bent, thereby causing unnecessary damage to the patient. Therefore, it is necessary to improve the bending resistance of the second curved section 112 while keeping the hardness of the second curved section 112 within a proper range, so as to ensure the safety of the delivery sheath during the implantation operation.

In some embodiments, the braided pattern of the braid 113 may affect not only the hardness of the second curved section 112, but also the fracture resistance of the second curved section 112. In some embodiments, the braiding method of the braid 113 is related to the shape, size, and braid density of the braided wire, and considering that the shape of the braided wire is not easy to change (such as round wire), the braiding method of the braid 113 is mainly related to the size (such as diameter) and braid density of the braided wire. For example, the higher the braid density, the greater the hardness; the lower the braid density, the lower the hardness. For example, the higher the braid density, the better the bending resistance; the lower the braid density, the worse the bending resistance.

In some embodiments, as shown in FIG. 3, when the delivery sheath is used for atrial septum pacing, for example, when the stereo curved shape of the first tube section 11 is D-type and E-type, the curved radius is smaller and the curving degree is higher, the second curved section 112 of the delivery sheath is also easier to bend than when the delivery sheath is used for interventricular septum, his bundle, and left bundle branch pacing, for example, when the stereo curved shape of the first tube section 11 is S-type, M-type, L-type, B-type and C-type. Based on this, when the delivery sheath is used for atrial septum pacing, for example, when the stereo curved shape of the first tube section 11 is D-type and E-type, the size (e.g. diameter) of the braided wire of the second curved section 112 may be appropriately increased and/or the braid density may be appropriately increased.

In some embodiments, when the delivery sheath is used for interventricular septum, his bundle and left bundle branch pacing, for example, the stereo curved shape of the first tube section 11 is S-type, M-type, L-type, B-type and C-type, the diameter of the braided wire of the second curved section 112 may range from 0.04 mm to 0.06 mm, and the braid density of the second curved section 112 may range from 50 to 70. In some embodiments, when the delivery sheath is used for interventricular septum, his bundle and left bundle branch pacing, for example, the stereo curved shape of the first tube section 11 is S-type, M-type, L-type, B-type and C-type, the diameter of the braided wire of the second curved section 112 may range from 0.05 mm to 0.07 mm, and the braid density of the second curved section 112 may range from 40 to 50. In some embodiments, when the delivery sheath is used for interventricular septum, his bundle and left bundle branch pacing, for example, the stereo curved shape of the first tube section 11 is S-type, M-type, L-type, B-type and C-type, the diameter of the braided wire of the second curved section 112 may range from 0.03 mm to 0.05 mm, and the braid density of the second curved section 112 may range from 70 to 80.

It will be appreciated that when the delivery sheath is used for interventricular septum, his bundle, and left bundle branch pacing, for example, when the stereo curved shape of the first tube section 11 is S-type, M-type, L-type, B-type, and C-type, by limiting the diameter and braid density of the braided wire of the braid 113 within a suitable range, not only can the hardness of the second curved section 112 be within a suitable range, but also can improve its resistance to fracture to a certain extent, thereby ensuring the safety of the delivery sheath during the implantation operation.

It should be noted that the diameter and the braid density of the braided wire of the second curved section 112 may be provided separately, or may be used in combination with the shape parameters (such as the curved radius of the second curved section 112) when the stereo curved shape of the first tube section 11 is S-shaped, M-shaped, L-shaped, B-shaped and C-shaped. It will be appreciated that, based on the shape parameters when the stereo curved shape of the first tube section 11 is S-shaped, M-shaped, L-shaped, B-shaped, and C-shaped, further defining the diameter range and braid density range of the braided wire of the second curved section 112, it is possible to further improve its resistance to buckling, thereby improving the safety of the delivery sheath during implantation. In some embodiments, the diameter range and braid density range of the braided wire of the second curved section 112 may be different when the shape parameters of the first tube section 11 are different as the stereo curved shape thereof is S-shaped, M-shaped, L-shaped, B-shaped and C-shaped. For example, when a curved radius of the second curved section 112 ranges from 18 cm to 35 cm, the braid density may range from 58 to 70. As another example, when the second curved section 112 has a curved radius ranging from 40 cm to 70 cm, the braid density may range from 50 to 55.

In some embodiments, when the delivery sheath is used for atrial septum pacing, for example, the stereo curved shape of the first tube section 11 is D-type and E-type, the diameter of the braided wire of the second curved section 112 may range from 0.05 mm to 0.07 mm, and the braid density of the second curved section 112 may range from 60 to 80. In some embodiments, when the delivery sheath is used for atrial septum pacing, for example, the stereo curved shape of the first tube section 11 is D-type and E-type, the diameter of the braided wire of the second curved section 112 may range from 0.06 mm to 0.08 mm, and the braid density of the second curved section 112 may range from 50 to 60. In some embodiments, when the delivery sheath is used for atrial septum pacing, for example, the stereo curved shape of the first tube section 11 is D-type and E-type, the diameter of the braided wire of the second curved section 112 may range from 0.03 mm to 0.05 mm, and the braid density of the second curved section 112 may range from 80 to 90.

It will be appreciated that when the delivery sheath is used for atrial septum pacing, for example, when the stereo curved shape of the first tube section 11 is D-type and E-type, by appropriately increasing the diameter and braid density of the braided wire of the braid 113, the bending resistance of the second curved section 112 is effectively improved while ensuring that the hardness of the second curved section 112 is within an appropriate range, thereby ensuring the safety of the delivery sheath during clinical operation.

It should be noted that the diameter and the braid density of the braided wire of the second curved section 112 may be provided separately, or may be used in combination with the shape parameters (such as the curved radius of the second curved section 112) when the stereo curved shape of the first tube section 11 is D-type and E-type as described above. It will be appreciated that the diameter range and braid density range of the braided wire of the second curved section 112 are defined based on the shape parameters of the first tube section 11 when the stereo curved shape is D-shaped and E-shaped, which further increases its resistance to buckling, thereby improving the safety of the delivery sheath during implantation. In some embodiments, when the shape parameters of the first tube section 11 are different as the stereo curved shape is D-shaped and E-shaped, the diameter range and braid density range of the braided wire of the second curved section 112 may be different. For example, when a curved radius of the second curved section 112 ranges from 10 cm to 12 cm, the braid density may range from 70 to 80. As another example, when the second curved section 112 has a curved radius ranging from 13 cm to 15 cm, the braid density may range from 60 to 70.

In some embodiments, the braided pattern of the braid 113 may also be related to the winding pattern of the braided wire. As described above, when the delivery sheath is used for atrial septum pacing, for example, when the stereo curved shape of the first tube section 11 is D-type and E-type, the curved radius is smaller and the curving degree is higher, and the second curved section 112 is also easier to bend than when the delivery sheath is used for interventricular septum, his bundle and left bundle branch pacing, for example, when the stereo curved shape of the first tube section 11 is S-type, M-type, L-type, B-type and C-type. Therefore, when the delivery sheath is used for interventricular septum, his bundle, and left bundle branch pacing, for example, when the stereo curved shape of the first tube section 11 is S-type, M-type, L-type, B-type and C-type, the second curved section 112 can be braided using two braided wires wound together as monofilaments (simply referred to as monofilament braiding), as shown in FIG. 5. When the delivery sheath is used for atrial septum pacing, for example, when the stereo curved shape of the first tube section 11 is D-type and E-type, the second curved section 112 may be knitted by using double-wire braid, i.e. the monofilaments with two braided wires wound together are knitted separately, as shown in FIG. 6. By using a double-wire braid, a natural transition in hardness can be achieved between different tube sections, thereby effectively improving the fracture resistance of the second curved section 112.

In some embodiments, the second tube section 12 and the third tube section 13 each include at least a braid 113, which is braided from braided wire. In some embodiments, the second tube section 12 and the third tube section 13 are similar in construction to the first tube section 11 and may further include an outer layer 114 and an inner layer 115. Further details regarding outer layer 114 and inner layer 115 can be found in the foregoing description.

In some embodiments, the hardness of each tube section of the sheath tube 1 is related not only to the material used for each tube section (e.g. the material used for the outer layer 114 and the inner layer 115) but also to the braided way of the braid 113. For example, the higher the braid density, the greater the hardness; the lower the braid density, the lower the hardness. In some embodiments, the braid density of the second tube section 12 may be less than the braid density of the third tube section 13 because the hardness of the second tube section 12 is less than the hardness of the third tube section 13.

In some embodiments, the braid density of the second tube section 12 ranges from 30 to 40, and the braid density of the third tube section 13 ranges from 50 to 70. In some embodiments, the braid density of the second tube section 12 ranges from 30 to 35, and the braid density of the third tube section 13 ranges from 50 to 60. In some embodiments, the braid density of the second tube section 12 ranges from 35 to 40, and the braid density of the third tube section 13 ranges from 60 to 70. In some embodiments, the braid density of the second tube section 12 ranges from 20 to 35, and the braid density of the third tube section 13 ranges from 40 to 60. In some embodiments, the braid density of the second tube section 12 ranges from 35 to 45, and the braid density of the third tube section 13 ranges from 60 to 75.

In some embodiments, the relationship between the braid densities of the second tube section 12 and the third tube section 13 may be expressed as a range of ratios of the braid densities. In some embodiments, the ratio of the braid density of the second tube section 12 to the braid density of the third tube section 13 ranges from 0.43 to 0.80. In some embodiments, the ratio of the braid density of the second tube section 12 to the braid density of the third tube section 13 ranges from 0.50 to 0.70. In some embodiments, the ratio of the braid density of the second tube section 12 to the braid density of the third tube section 13 ranges from 0.55 to 0.75. In some embodiments, the ratio of the braid density of the second tube section 12 to the braid density of the third tube section 13 ranges from 0.40 to 0.55. In some embodiments, the ratio of the braid density of the second tube section 12 to the braid density of the third tube section 13 ranges from 0.70 to 0.85.

According to some embodiments of the present specification, by defining the range of the braid density or the range of the ratio of the braid densities of the second tube section 12 and the third tube section 13, the hardness of the second tube section 12 and the third tube section 13 can be within a suitable hardness range, and at the same time, the resistance to bending can be improved to a certain extent, so as to ensure the pushing performance and safety of the delivery sheath.

FIG. 7 is a schematic view of the structure of a connection between the sheath base and the sheath tube according to some embodiments of the present specification; FIG. 8 is a schematic view of the structure of a connection between the sheath base and the sheath tube according to further embodiments of the present specification. As shown in FIG. 7 and FIG. 8, the delivery sheath further includes a sheath base 2, and the sheath tube 1 further includes a cut tube section 14 provided at a proximal end and an implant tube section 15 provided at a distal end of the cut tube section 14, the first tube section 11 is located at a distal end of the implant tube section 15.

The implant tube section 15 refers to the tube section of the sheath tube 1 at the distal end of the cut tube section 14. In some embodiments, the implant tube section 15 may include a first tube section 11, a second tube section 12, and a third tube section 13. In some embodiments, the implant tube section 15 may also include other tube sections. In some embodiments, the implant tube section 15 is used to access a medical device, such as an electrode lead, and guide it to a target lumen.

The cut tube section 14 refers to the transition tube section of the sheath tube 1 for connection to the sheath base 2. In some embodiments, the cut tube section 14 includes a connecting region 141 and a buffer region 142 provided from proximal to distal along the axial direction of the sheath tube 1, the sheath tube 1 is connected to the sheath base 2 by the connecting region 141, the buffer region 142 is configured to provide cutting resistance between the sheath base 2 and the implant tube section 15. In some embodiments, the axial direction of the sheath tube 1 may be indicated by the direction X indicated by the arrow in FIG. 7. From near to far means from one end close to the operator to one end far away from the operator.

Connecting region 141 refers to the cut tube section 14 that is used for connecting to the sheath base 2. In some embodiments, the connecting region 141 is located within the sheath base 2 (e.g. within the channel 231 described hereinafter) and is connected to the sheath base 2 in a variety of ways. Exemplary attachment means include, but are not limited to, clamping, bonding, etc. The buffer region 142 refers to the transition tube section between the connecting region 141 and the implant tube section 15. In some embodiments, the buffer region 142 may be used to provide cutting resistance.

As described above, after the fixation of the electrode is completed, it is usually necessary to withdraw the sheath tube 1 from the body, but since the other end connected with the electrode is provided with other test equipment, it will interfere with the withdrawal of the sheath, so it is necessary to cut the sheath tube 1 along its axial direction to complete the whole withdrawal of the sheath. Cutting resistance refers to the resistance experienced by an operator (such as a physician) when cutting the sheath tube 1 in the axial direction of the sheath tube 1 and withdrawing the sheath tube 1 out of the body.

In some embodiments, the cutting resistance provided by the buffer region 142 decreases from proximal to distal along the axial direction of the sheath tube 1. It will be appreciated that when the operator cuts the sheath tube 1 along the axial direction of the sheath tube 1, a part of the sheath tube section (such as the connecting region 141 of the cut tube section 14) located in the sheath base 2 has a greater cutting resistance due to being blocked by the sheath base 2 so that a greater cutting force can be used for cutting; Since the sheath tube section located outside the sheath base 2 loses the blocking of the sheath base 2, the cutting resistance will suddenly decrease, so it is necessary to use a smaller cutting force for cutting to avoid scratching the patient. Therefore, by making the cutting resistance provided by the transition tube section (such as the buffer region 142) provided in the sheath base 2 decrease from proximal to distal along the axial direction of the sheath tube 1, it is possible to avoid the abrupt change of the cutting force used in the cutting, so that the whole cutting process is smoother, thereby reducing the operational difficulty of the operator and the risk of the surgery, and improving the safety of the surgery.

In some embodiments, the structure of the buffer region 142 may be designed in order to make the cutting resistance provided by the buffer region 142 decrease from proximal to distal along the axial direction of the sheath tube 1, so as to make the whole cutting process smoother, thereby reducing the operational difficulty of the operator and the risk of the surgery and improving the safety of the surgery.

In some embodiments, as shown in FIG. 7, the buffer region 142 includes a first raised structure 1421 provided on an inner wall of the buffer region 142. The first raised structure 1421 refers to a raised structure provided on the inner wall of the buffer region 142 for increasing cutting resistance. The first raised structure 1421 may be designed in various structural shapes such as a rectangular parallelepiped shape or the like. In some embodiments, the first raised structure 1421 may be integrally formed with the buffer region 142 or may be bonded to the inner wall of the buffer region 142.

In some embodiments, the thickness of the first raised structure 1421 decreases from proximal to distal along the axial direction of the sheath tube 1. Wherein the thickness of the first raised structure 1421 refers to the wall thickness of the first raised structure 1421 in the radial direction of the sheath tube 1. It will be appreciated that since the thickness of the first raised structure 1421 decreases from proximal to distal along the axial direction of the sheath tube 1, the cutting resistance provided by the first raised structure 1421 provided on the inner wall of the buffer region 142 also decreases from proximal to distal along the axial direction of the sheath tube 1, so that the cutting resistance provided by the buffer region 142 decreases from proximal to distal along the axial direction of the sheath tube 1, effectively avoiding abrupt changes in cutting force during cutting.

In some embodiments, the first raised structure 1421 includes a cutting guide slot 14211, the width of which decreases from proximal to distal along the axial direction of the sheath tube 1. The cutting guide slot 14211 refers to a groove structure opened along the axial direction of the sheath tube 1 on the first raised structure 1421, such as a V-shaped groove. The width of the cutting guide slot 14211 refers to the width of the cutting guide slot 14211 in the circumferential direction of the sheath tube 1. In some embodiments, the cutting guide slot 14211 is opened in the middle of the first raised structure 1421 for guiding the cutting direction of the cutting blade. It will be appreciated that the width of the cutting guide slot 14211 decreases from proximal to distal along the axial direction of the sheath tube 1, and it is more convenient for the operator to perform the cutting operation while guiding the cutting blade.

It should be noted that the depth of the cutting guide slot 14211 is less than the thickness of the first raised structure 1421 to ensure that the cutting force required to cut the area of the buffer region 142 where the first raised structure 1421 is provided is greater than the cutting force required to cut the area of the buffer region 142 where the first raised structure 1421 is not provided. In some embodiments, the depth of the cutting guide slot 14211 may decrease from proximal to distal along the axial direction of the sheath tube 1.

In some embodiments, the first raised structure 1421 includes a plurality of raised portions that are spaced along an axial direction of the sheath tube 1. As shown in FIG. 7, each raised portion has a cutting guide slot 14211, and the thickness and/or length of the plurality of raised portions decreases from proximal to distal along the axial direction of the sheath tube 1. Wherein the thickness of the raised portion refers to the wall thickness of the raised portion in the radial direction of the sheath tube 1; the length of the raised portion means the length of the raised portion in the circumferential direction of the sheath tube 1. It should be noted that the quantity of the plurality of raised portions is not limited, and may be provided according to actual requirements. In some embodiments, the first raised structure 1421 may also include only one raised portion, i.e. one raised portion is the first raised structure 1421.

In some embodiments, as shown in FIG. 7, the buffer region 142 includes a second raised structure 1422 provided on the inner wall of the buffer region 142, and the thickness of the second raised structure 1422 decreases from proximal to distal along the axial direction of the sheath tube 1. The second raised structure 1422 refers to a raised structure provided on the outer wall of the buffer region 142 for increasing cutting resistance. In some embodiments, the second raised structure 1422 may be integrally formed with the buffer region 142 or may be bonded to the outer wall of the buffer region 142 by bonding or the like.

In some embodiments, the second raised structure 1422 may include a first raised portion 14221 and a second raised portion 14222, the first raised portion 14221 and the second raised portion 14222 being symmetrically provided. In some embodiments, a gap is provided between the first raised portion 14221 and the second raised portion 14222 for providing frictional resistance to the cutting blade, the gap is flared, and the width of the gap decreases from proximal to distal along the axial direction of the sheath tube 1. In some embodiments, the portion of the gap close to the connecting region 141 has a greater width that may be used to guide the cutting blade; the smaller width of the portion of the gap close to the implant tube section 15 increases cutting resistance. This structure is designed to increase the friction and resistance between the cutting blade and the sheath tube 1 in buffer region 142 so that the cutting force used by the operator when cutting the buffer region 142 is gradually reduced, thus effectively eliminating the mutation of cutting force in the cutting process, making the whole cutting process smoother, so as to reduce the operator's operation difficulty and surgical risk and avoid unnecessary injury to the patient.

It should be noted that the buffer region 142 may be provided with both the first raised structure 1421 and the second raised structure 1422, or may be provided with only the first raised structure 1421 or the second raised structure 1422. When the buffer region 142 is provided with both the first raised structure 1421 and the second raised structure 1422, the cutting guide slot 14211 of the first raised structure 1421 is collinear with the gap of the second raised structure 1422. It will be appreciated that since both the gap and the cutting guide slot 14211 function to guide the cutting path of the cutting blade, designing the gap and the cutting guide slot 14211 to be collinear can make it unnecessary for the operator to change the cutting path when performing the cutting operation, improving the convenience of the operation.

It should be noted that the first raised structure 1421 and the second raised structure 1422 are provided in various manners. In some embodiments, the first raised structure 1421 and the second raised structure 1422 may be provided on an inner wall of the buffer region 142 at the same time (as shown in FIG. 7), or on an outer wall of the buffer region 142 at the same time (as shown in FIG. 8). In some embodiments, the first raised structure 1421 and the second raised structure 1422 may also be provided on an inner wall and an outer wall of the buffer region 142, respectively.

FIG. 9 is a schematic view of the structure of a sheath base according to some embodiments of the present specification; FIG. 10 is a schematic view of the structure that a sheath base body connected to a handle according to some embodiments of the present specification. As shown in FIG. 9 and FIG. 10, the sheath base 2 includes a side branch 21, a handle 22, and a sheath base body 23, wherein the handle 22 is connected to the sheath base body 23, one end of the side branch 21 passes through the handle 22, and communicates with the sheath base body 23, and the other end of the side branch 21 is provided with a valve 211, and at least one joint (not shown in the figures) is connected to the valve 211.

The side branch 21 is a tubular structure of the sheath base 2 for enabling the sheath tube 1 to communicate with other devices or equipment. Connectors are connectors, including but not limited to luer connectors, etc. In some embodiments, a joint may be used to communicate the side branch 21 with other devices or equipment. For example, a connector may be attached to the aspiration device to allow aspiration of air or infusion of anticoagulants such as heparin. The valve 211 is a control member, including but not limited to a three-way valve or the like. In some embodiments, valve 211 may control the communication and disconnection of side branch 21 from other devices or equipment. When valve 211 is closed, air is prevented from entering the blood vessel and blood is prevented from flowing out of the body.

The handle 22 is a structure for an operator to hold the sheath base 2, and the operator can perform a cardiac pacemaker implantation operation by holding the handle 22. In some embodiments, the handle 22 has a good hand and grip to help the operator better grasp and use the delivery sheath. In some embodiments, the handle 21 and the sheath base body 23 may be integrally formed or may be attached by welding, adhesive, etc.

In some embodiments, as shown in FIG. 10, the sheath base body 23 is provided with a channel 231 in the axial direction of the sheath tube 1, and one end of the side branch 21 communicates with the channel 231, which is configured to deliver a medical device. In some embodiments, the sheath base body 23 has a thin-walled structure 232 in the axial direction of the sheath tube 1, and the sidewall of the sheath base body 23 is provided with a clamping block 233.

The channel 231 is a pathway for the sheath base body 23 to deliver a medical device such as electrode leads. In some embodiments, the connecting region 141 of the sheath tube 1 is provided within a channel 231 through which the medical device may be introduced into the sheath tube 1 for delivery into the target lumen. In some embodiments, the channel 231 may be sized according to the dimensions of the sheath base body 23 and the medical device.

The thin-walled structure 232 is part of the sidewall of the sheath base body 23. In some embodiments, the thickness of the thin-walled structure 232 decreases from proximal to distal along the axial direction of the sheath tube 1, thereby gradually reducing the cutting force required to cut the sheath base body 23 for better cutting by the operator. The thickness of the thin-walled structure 232 refers to the thickness of the thin-walled structure 232 in the radial direction of the sheath tube 1.

The clamping block 233 is a structure provided on the sidewall of the sheath base body 23 for connecting the sheath cap. In some embodiments, the quantity of the clamping blocks 233 may be one or more, and when the quantity of the clamping blocks 233 is one, the clamping blocks 233 may be looped around the sidewall of the sheath base body 23; when the quantity of the clamping blocks 233 is plural, the clamping blocks 233 may be symmetrically provided on the sidewall of the sheath base body 23. In some embodiments, the clamping block 233 may be integrally formed with the sheath base body 23 or may be attached by bonding, clamping, or the like. For further details of the sheath cap, reference is made to FIG. 11 and the associated description thereof.

In some embodiments, the sheath base body 23 is further provided with a cut-out (not shown) in the axial direction of the sheath tube 1, the cut-out extending from the proximal end of the sheath base body 23 to the distal end of the sheath base body 23 for cutting guidance of the cutting blade. In some embodiments, the cutout in the sheath base body 23, the cutting guide slot 14211 of the first raised structure 1421, and/or the gap of the second raised structure 1422 are collinear, and the cutting tip naturally transitions from the cutout to the cutting guide slot 14211 and/or the gap when cutting, improving the guiding effect for the cutting blade.

FIG. 11 is a schematic view of the structure of a sheath cap according to some embodiments of the present specification. As shown in FIG. 11, the delivery sheath further includes a sheath cap 3 provided with a device channel 31 along the axial direction of the sheath tube 1, and a sidewall of the sheath cap 3 is provided with a clamping slot 32 which is clamped with a clamping block 233 of the sheath base body 23.

The sheath cap 3 is a structure covering the proximal end of the sheath base body 23. In some embodiments, the sheath cap 3 may be clamped with the clamping block 233 of the sheath base body 23 through the clamping slot 32 to cover the proximal end of the sheath base body 23. In some embodiments, the sheath cap 3 may include a sheath cap top and an annular sidewall connecting the sheath cap top, with the clamping slot 32 opening into the annular sidewall. In some embodiments, the sheath cap 3 may be used to secure a hemostasis valve. For further details of the hemostasis valve 4, reference is made to FIG. 12 to FIG. 14 and the associated description thereof.

The clamping slot 32 is provided on the sidewall of the sheath cap 3 for connecting to the sheath base 2. In some embodiments, the clamping slot 32 is provided corresponding to the clamping block 233 of the sheath base body 23, and the connection between the sheath cap 3 and the sheath base 2 (such as the sheath base body 23) can be realized by clamping the clamping block 233 of the sheath base body 23 in the clamping slot 32. The device channel 31 is the pathway through which the sheath cap 3 is used to pass medical devices such as electrode leads. In some embodiments, device channel 31 at least partially coincides with channel 231 of sheath base body 23 when sheath cap 3 is provided over the proximal end of sheath base body 23. In some embodiments, the device channel 31 extends in the radial direction of the sheath tube 1 toward one side of the sheath cap 3 to form a notch designed to allow an operator to cut the sheath base body 23 without cutting the sheath cap 3, making it more time and effort efficient.

FIG. 12 is a schematic view of the structure of a hemostasis valve according to some embodiments of the present specification; FIG. 13 is a schematic view of the structure of opening an outlet of a hemostasis valve according to some embodiments of the present specification; FIG. 14 is a schematic view of the structure of closing an outlet of a hemostasis valve according to some embodiments of the present specification. As shown in FIG. 12 to FIG. 14, the delivery sheath further includes a hemostasis valve 4 provided within a channel 231 of the sheath base body 23. The hemostasis valve 4 is provided with a circular hole structure 41 for the passage of medical devices and an elastic structure 42 provided at the distal end of the circular hole structure 41 and configured to open or close the circular hole structure 41.

The hemostasis valve 4 is a structure for preventing blood in a blood vessel from flowing out. The material of the hemostasis valve 4 may include but is not limited to, silicone, etc.

The circular hole structure 41 is a hole structure on the hemostasis valve 4 for providing access to the medical device. In some embodiments, the circular hole structure 41 may include a first circular hole 411 at the proximal end of the hemostasis valve 4 and a second circular hole 412 at the distal end of the hemostasis valve 4, the first circular hole 411 communicating with the second circular hole 412. Medical devices (such as dilators, electrode guide wires, etc.) can be introduced into the sheath tube 1 through the first circular hole 411 and the second circular hole 412 into the channel 231 of the sheath base body 23 in sequence. Here, the first circular hole 411 can be understood as an inlet of the hemostasis valve 4, the second circular hole 412 can be understood as an outlet of the hemostasis valve 4, and the inner diameter of the first circular hole 411 is greater than the inner diameter of the second circular hole 412. In some embodiments, the first circular hole 411 is offset provided from the second circular hole 412 to facilitate better hemostasis.

The elastic structure 42 is a sheet-like structure on the hemostasis valve 4 for opening or closing the circular hole structure 41. For example, the elastic structure 42 may include an elastic sheet or the like. In some embodiments, the elastic structure 42 may be automatically closed under its own action. Since the elastic structure 42 is provided at the distal end of the circular hole structure 41 (e.g. the second circular hole 412), and the opening is oriented close to the sheath tube 1 when it is opened, the elastic structure 42 only allows the medical device to be inserted into the sheath tube 1, and reasonable hemostasis can be achieved without affecting the insertion of the medical device such as a dilator. When the medical device is inserted, the elastic structure 42 opens (as shown in FIG. 13) and hemostasis is achieved by the medical device in an interference fit with the second circular hole 412. When the medical device is withdrawn, the elastic structure 42 may automatically close under its own action (as shown in FIG. 14) to achieve hemostasis. That is, regardless of whether a medical device passes through the circular hole structure 41 (e.g. the second circular hole 412), the hemostasis valve 4 can maintain a seal, thereby achieving a hemostatic effect.

In some embodiments, as shown in FIG. 12, the hemostasis valve 4 is further provided with reinforcing ribs 43 provided around the outer periphery of the hemostasis valve 4 at a position close to the elastic structure 42. In some embodiments, the reinforcing rib 43 may not only better assist in automatically closing the elastic structure 42, but may also better achieve a hemostatic function when the medical device is withdrawn. At the same time, the reinforcing ribs 43 can also prevent the elastic structure 42 from opening when negative pressure is clinically drawn, resulting in air entering the blood vessel.

It should be noted that the hemostasis valve 4 may be provided within the channel 231 of the sheath base body 23 in a variety of ways. In some embodiments, the hemostasis valve 4 may be secured within the channel 231 of the sheath base body 23 by bonding, welding, clamping, or mechanical positioning. In some embodiments, the hemostasis valve 4 may be secured within the channel 231 of the sheath base body 23 by the sheath cap 3. By way of example only, the hemostasis valve 4 has an outer edge extending in the radial direction of the sheath tube 1, the hemostasis valve 4 is located in the channel 231 of the sheath base body 23, the outer edge of the hemostasis valve 4 is clamped between the device channel 31 of the sheath cap 3 and the sheath cap top, and when the clamping block 233 on the sheath base body 23 is inserted into the clamping slot 32 of the sheath cap 3, the hemostasis valve 4 is fixed in the channel 231 of the sheath base body 23.

Benefits that may be provided by embodiments of the present specification include, but are not limited to: 1) by designing the structure and shape of the sheath tube (such as the first tube section), it is possible to provide a whole series of delivery sheaths for pacing at different sites, so as to solve the problem of great difficulty in clinical operation due to the difference in size of chambers of the patient's heart, reduce the operation difficulty of operators, and solve the problem of no suitable delivery sheath in the field of atrial septum pacing; 2) by providing a raised structure (such as a first raised structure and a second raised structure) on the cut tube section, the cutting can be made smoother, thereby reducing the operating difficulty of the operator and the surgical risk, and effectively avoiding possible injury to the patient; 3) by providing the hemostasis valve, air can be prevented from entering blood vessels when negative pressure is clinically applied, and the hemostasis valve can be closed by itself when the medical device is pulled out, so that the hemostasis effect is better and more reliable.

The basic concepts have been described above; it should be apparent to those skilled in the art that the foregoing detailed disclosure is by way of example only and is not to be construed as limiting the present specification. While not explicitly described herein, various modifications, improvements, and adaptations to the present specification may occur to those skilled in the art. Such alterations, improvements, and modifications are intended to be suggested herein and are intended to be within the spirit and scope of the exemplary embodiments of the present specification.

1-27. (canceled)

28. A cardiac pacemaker electrode lead delivery sheath, comprising:
a sheath base, provided with a sheath tube interface;
a sheath tube, comprising a cut tube section provided in sequence along an axial direction of the sheath tube and an implant tube section disposed at a distal end of the cut tube section, the cut tube section comprising a connecting region and a buffer region provided in sequence along the axial direction, the connecting region being fixedly connected to the sheath tube interface, the buffer region connecting the connecting region and the implant tube section, the buffer region being configured to provide a cutting resistance between the sheath base and the implant tube section, and the magnitude of cutting resistance provided by the buffer region being between the magnitude of cutting resistance provided by the sheath base and the implant tube section.

29. The cardiac pacemaker electrode lead delivery sheath according to claim 28, wherein the buffer region provides a cutting resistance that decreases in magnitude from a side close to the connecting region to a side close to the implant tube section.

30. The cardiac pacemaker electrode lead delivery sheath according to claim 29, wherein an inner wall of the buffer region is provided with a raised structure such that the buffer region provides the cutting resistance.

31. The cardiac pacemaker electrode lead delivery sheath according to claim 30, wherein the raised structure comprises a first raised structure capable of being cut when the cutting blade cuts the buffer region, the first raised structure extends in a circumferential direction of an inner wall of the sheath tube, and a thickness of the first raised structure decreases from a side close to the connecting region to a side close to the implant region.

32. The cardiac pacemaker electrode lead delivery sheath according to claim 31, wherein
a quantity of the first raised structures is one or more;
when the quantity of the first raised structures is plural, a plurality of the first raised structures are spaced along the axial direction of the sheath tube, and the thickness and/or length of the first raised structures close to the connecting region is greater than the thickness and/or length of the first raised structures close to the implant tube section.

33. The cardiac pacemaker electrode lead delivery sheath according to claim 31, wherein the first raised structure has a groove for guiding the cutting direction of the cutting blade; the groove opens along the axial direction of the sheath tube and is recessed from the surface of the first raised structure toward the inner wall of the sheath tube.

34. The cardiac pacemaker electrode lead delivery sheath according to claim 29, wherein the raised structure comprises a second raised structure comprising a first raised portion and a second raised portion, the first raised portion and the second raised portion having a gap therebetween, the gap having a width configured to grip a cutting blade to provide frictional resistance to the cutting tool to cut the buffer region.

35. The cardiac pacemaker electrode lead delivery sheath according to claim 34, wherein a width of the gap increases from a side close to the connecting region to a side close to the implant tube section; and/or the portion of the gap close to the connecting region forms a flare.

36. The cardiac pacemaker electrode lead delivery sheath according to claim 34, wherein
a quantity of the second raised structures is one or more;
when the quantity of the second raised structures is plural, a plurality of the second raised structures are provided along the circumferential direction of the inner wall of the sheath tube.

37. The cardiac pacemaker electrode lead delivery sheath according to claim 29, wherein the sheath tube is formed by machining a plurality of sections of high polymer materials of different hardness in an axial direction, and the hardness of the sheath tube decreases from a proximal end close to the sheath base to a distal end far from the sheath base.

38. The cardiac pacemaker electrode lead delivery sheath according to claim 28, wherein the sheath base comprises a sheath base body, a side branch, and a handle, the sheath base body is provided with a sheath cap interface, the sheath tube interface and a channel communicating the sheath tube interface and the sheath cap interface, the handle is connected to the sheath base body, and the side branch passes through the handle and communicates with the passage; the wall thickness on the side of the sheath tube body close to the sheath tube interface is less than the wall thickness on the side close to the sheath cap interface.

39. The cardiac pacemaker electrode lead delivery sheath according to claim 38, characterized by further comprising a hemostasis valve, an inlet of the hemostasis valve being provided at the sheath cap interface, and an outlet of the hemostasis valve passing through the sheath cap interface into the channel; the hemostasis valve being provided with an elastic structure for covering the outlet and the elastic structure can be pushed open by a pushing force to expose the outlet, and being automatically closed to close the outlet after the pushing force being released; a reinforcing rib being provided on the outer periphery of the hemostasis valve close to the elastic structure.

40. The cardiac pacemaker electrode lead delivery sheath according to claim 41, wherein the hemostasis valve is fixed at the sheath cap interface via a sheath cap, the sheath cap comprises a sheath cap body, a bayonet, and a device channel, the sheath cap body comprises a sheath cap top and an annular sidewall connected to the sheath cap top, the bayonet opens on the annular sidewall, and the device channel extends from the sheath cap top to the annular sidewall; the outer wall of the sheath base body is provided with a clamping block, the outer edge of the inlet of the hemostasis valve abuts between the sheath cap interface and the sheath cap top, and the clamping block is clamped into the bayonet of the sheath cap.

41. The cardiac pacemaker electrode lead delivery sheath according to claim 28, wherein the sheath tube comprises at least a first tube section provided at a distal end of the implant tube section, the first tube section has a first curved section and a second curved section, the first curved section is provided at a distal end of the first tube section; wherein the included angle between the first bending surface of the first curved section and the second bending surface of the second curved section is not less than 45°, and the hardness of the first curved section is less than the hardness of the second curved section.

42. The cardiac pacemaker electrode lead delivery sheath according to claim 41, wherein the first tube section comprises at least a braid, the braid is made from braided wires, a diameter of the braided wire of the second curved section ranges from 0.04 mm to 0.06 mm and a braid density of the second curved section ranges from 50 to 70.

43. A cardiac pacemaker electrode lead delivery sheath, comprising:
a sheath base, provided with a sheath tube interface;
a sheath tube, comprising a cut tube section provided in sequence along an axial direction of the sheath tube and an implant tube section disposed at a distal end of the cut tube section, the cut tube section comprising a connecting region and a buffer region provided in sequence along an axial direction, the connecting region being fixedly connected to the sheath tube interface, the buffer region connecting the connecting region and the implant tube section, the inner wall of the buffer region being provided with a raised structure, and the raised structure enables the magnitude of the cutting resistance provided by the buffer region to be between the magnitude of the cutting resistance provided by the sheath base and the implant region;
wherein
the raised structure comprises a first raised structure and a second raised structure, the first raised structure extending in a circumferential direction of an inner wall of the sheath tube and capable of being cut when the cutting blade cuts the buffer region; the second raised structure comprising a first raised portion and a second raised portion, the first raised portion and the second raised portion having a gap therebetween, the gap having a width configured to grip a cutting blade to provide frictional resistance to the cutting tool to cut the buffer region.

44. The cardiac pacemaker electrode lead delivery sheath according to claim 43, wherein the first raised structure has a groove for guiding the cutting direction of the cutting blade, the groove corresponding to the gap.

45. A preparation method for the cardiac pacemaker electrode lead delivery sheath according to claim 28, wherein
when an extruder is used to make a sheath tube, the head end of the extruder is subjected to stepwise temperature control, wherein the stepwise temperature control means that the extrusion temperature for making the part with a larger wall thickness on the sheath tube is higher than the extrusion temperature for making the part with a smaller wall thickness on the sheath tube.

46. The preparation method for the cardiac pacemaker electrode lead delivery sheath according to claim 45, wherein
the temperature difference between the extrusion temperature for making the part with a larger wall thickness on the sheath tube and the extrusion temperature for making the part with a smaller wall thickness on the sheath tube is determined according to the thickness difference between the part with a larger wall thickness on the sheath tube and the part with a smaller wall thickness on the sheath tube, the temperature difference increasing by 8° C.-10° C. for every 1 mm increase in the thickness difference.

47. The preparation method for the cardiac pacemaker electrode lead delivery sheath according to claim 45, wherein
a plurality of hole-shape exhaust structures are further provided on the die at the head end of the extruder.
